(19) United States
(12) Patent Application Publication
(10) Pub. No.: US 2005/0260263 A1
(43) Pub. Date: Nov. 24, 2005

(54) Sustained release formulation for sparingly soluble main drugs

(75) Inventors: Yih Ming Hsiao, Taipei (TW); Te Yu Liu, Jongli City (TW); Ming Jung Lin, Changhua Hsien (TW); Ling Feng Hsiao, Taoyuan Hsien (TW); Chung-Ming Chiang, Taipei (TW)

Correspondence Address: SCULLY SCOTT MURPHY & PRESSER, PC, 400 GARDEN CITY PLAZA, SUITE 300, GARDEN CITY, NY, 11530, US

(73) Assignee: Panion & BF Biotech Inc., Taipei (TW)

(21) Appl. No.: 10/848,020

(22) Filed: May 18, 2004

(51) Int. Cl. A61K 9/22; A61K 31/7048

(52) U.S. Cl. 424/468; 514/29

(57) ABSTRACT

A sustained release, solid pharmaceutical preparation for oral use in a daily dosage regimen is provided, which comprises at least one sparingly soluble main drug, such as an erythromycin derivative. The pharmaceutical preparation comprises the main drug(s), a water-soluble alginate salt and an organic carboxylic acid. In particular, a controlled release tablets of clarithromycin is provided.

The present invention relates to sustained release oral dosage forms of sparingly soluble main drugs, such as erythromycin derivatives, suitable to be digested in the gastriointestinal fluid. The present invention relates more particularly to a pharmaceutical composition of clarithromycin which is in the form of tablet to be administered once a day.

### BACKGROUND OF THE INVENTION

Erythromycin and its derivatives are antibacterial agents. They have a relatively broad spectrum of activity that overlaps the activity of penicillin. They are more effective against gram-positive cocci, such as enterococci, Group A hemolytic streptococci, pneumococci and staphaureus. They can be taken two to four times a clay, depending on the dosage, for a regimen of 10 to 14 days. The regimen of these two to four times daily dosage forms may cause inconvenience for the outpatients to handle the tablets and they may possibly forget to take the tablets when required. Therefore, it needs to reduce dosage regimens. Sustained release solid formulations have allowed the possibility of reducing dosage regimens for drugs, especially to outpatients. In addition, sustained release solid dosage forms can maintain steady-state plasma concentration-time profiles in the human to avoid possibly the overdose of using the two to four times daily dosage forms after the next tablet is taken.

It has been known that an alginate gel is utilized to prepare the controlled release tablets. A water soluble alginate such as sodium alginate is reacted with a calcium salt to convert it into an insoluble calcium alginate gel. The release-controlling character of the alginate gel depends on the molecular weight of alginate, the alginate concentration, the type of the polyvalent cation cross-linking agent, and/or the concentration of the cation. The use of a mixture of sodium alginate and sodium-calcium alginate for preparing controlled release solid dosage forms has been is disclosed in U.S. Pat. No. 4,842,866.

The technology of the above-mentioned patent could not be applied to generate formulations containing sparingly water-soluble drugs, such as an alginate-based formulation containing clarithromycin, which was observed to have too low dissolution rates and whose bioavailablity do not have reproducibility in the in vivo animal studies. The controlled release solid dosage forms for the sparingly water-soluble drugs can be prepared by using an alginate matrix with the incorporation of an organic acid. U.S. Pat. No. 5,705,190 (corresponding to Taiwan Patent No 429154) discloses a controlled release solid pharmaceutical composition comprising a water-soluble alginate salt, a complex salt of alginic acid and an organic carboxylic acid to facilitate dissolution of the active drugs. An example of the active drug is clarithromycin, which is very soluble in the gastric fluid and fairly soluble in the upper region of the small intestine (pH 5.0) where absorption is most likely to occur, but is poorly soluble in the region of the lower intestine (with pH 6 to 8). In order to increase the solubility of clarithromycin in the region of the lower intestine, citric acid is used in the formulation. In the pharmaceutical composition for the oral administration as claimed in U.S. Pat. No. 5,705,190, the weight ratio of sodium alginate to sodium-calcium alginate is about 16:1 to 1:1, and the molar ratio of organic acid (such as citric acid) to main drug is about 1:1.

The controlled release solid pharmaceutical compositions disclosed in U.S. Pat. No. 5,705,190 contain sodium alginate, calcium-sodium alginate and organic carboxylic acid (for example citric acid). However, these controlled release compositions do not purport to minimize the adverse effects related to gastrointestinal disorders including nausea and vomiting and a phenomenon described as taste perversion. To overcome some of these problems, improved controlled release formulations for the sparingly soluble main drugs such as erythromycin, have been developed in U.S. Pat. No. 6,010,718. These formulations contain a pharmaceutically acceptable polymer such as hydroxypropylmethyl cellulose to improve taste profile and reduce gastrointestinal side effects as compared to those for the immediate release composition. U.S. Pat. No. 6,551,616 B1 is similar to U.S. Pat. No. 6,010,718. The former patent contains more study results of gastrointestinal adverse effects in patients and indicates that patients taking the extended-release formulation of clarithromycin were significantly less likely to stop taking clarithromycin due to gastrointestinal adverse events and these patients suffered significantly fewer severe gastrointestinal adverse effects.

As in U.S. Pat. No. 5,705,190, Klaricid® XL Tablets of Abbott Laboratories Ltd., Queenborough, Kent, ME11 5EL UK (clarithromycin extended release tablets). contain 500 mg of clarithromycin and the following inactive ingredients: citric acid, sodium alginate, sodium-calcium alginate, lactose, povidone, talc, stearic acid, magnesium stearate, methyl hydroxypropyl cellulose, polyethylene glycol, titanium dioxide (E171), sorbic acid and quinoline yellow dye (E104). This formula contains sodium alginate (a water-soluble alginate salt), sodium-calcium alginate (a water-insoluble alginate salt) and citric acid (an organic carboxylic acid) to facilitate the dissolution of clarithromycin. Sodium alginate and sodium-calcium alginate are difficult to mix well because of the big difference of their amounts in the formula. In Klaricid® XL Tablets the weight ratio of sodium alginate to sodium-calcium alginate is about 8:1, though it is mentioned in U.S. Pat. No. 5,705,190 that the ratio is about 16:1 to 1:1. If the contents of sodium alginate and sodium-calcium alginate are similar such that their ratio is 2:1 or 1:1, the dissolution of clarithromycin would be much slower than current requirement in the bioequivalence study.

### OUTLINE OF THE INVENTION

The object of the present invention is to provide a controlled release pharmaceutical formulation of sparingly soluble main drug, such as erythromycin derivatives, for oral use. It is sufficient enough to use only sodium alginate instead of the combination of sodium alginate and calcium-sodium in the controlled release formulation. Sodium alginate is useful for forming a viscous colloidal solution and is insoluble in acidic solutions having pH of less than 3, although it is soluble in water. Therefore, sodium alginate can protect erythromycin derivatives from quick dissolution in the gastric fluid, and thereby improving the stability of erythromycin derivatives. Also, using only sodium alginate in the controlled release formulation can avoid the problem that in the manufacturing process the components are difficult to mix well.

In another aspect, the present invention provides a controlled release pharmaceutical composition for oral use, comprising sparingly soluble drugs, such as erythromycin derivatives, and a film coating. Upon comparison with immediate release formulations, the composition of the present invention has an improved taste profile and minimized adverse effects related to gastrointestinal disorders.

### DETAILED DESCRIPTIONS OF THE INVENTION

The present invention provides a once a day dose regimen for sparingly soluble drugs, such as erythromycin derivatives, by giving a controlled release, solid pharmaceutical composition for oral administration to a patient in need thereof. A preferred dosage form is “tablet”.

Erythromycin is one of the macrolide antibiotics. It is soluble in alcohol and in other common organic solvents but only slightly soluble in water. It is extremely unstable at a pH of 4 or lower. Erythromycin derivatives may include the following types: an acid addition salt of the dimethylamino group on the desoamine moiety, such as glucoheptonate, lactobionate and stearate; and an ester of the OH group on the desoamine moiety such as ester of ethylcarbonate, ethylsuccinate and propionate; and clarithromycin.

The pharmaceutical composition of the present invention may include other drugs combined with erythromycin derivatives wherever known combination therapy is required or beneficial. These other drugs related to gastritis, ulcers or gastroesophageal reflux disease (GERD) are anti-ulcer or anti-gastritis medicaments, selected among gastric secretion inhibiting compounds such as sulfamethoxazole, metronidazole, cimetidine, indapamide, atenolol, diazepam, omeprazole, ranitidine, sulcralfate, etc., or antacids such as magnesium hydroxide, aluminum hydroxide, sodium carbonate, simethicone, etc..

The amount(s) of drug(s) in the pharmaceutical composition may vary from about 20 to 90% of the total composition or tablet. For clarithromycin, the amount may preferably vary from 40% to 80% of the weight of the total composition or tablet.

While sodium alginate is preferably used in the present invention, other cations, such as potassium, ammonium or other alkaline metal cations, may replace sodium cation to from a soluble alginate salt. The amount of the soluble alginate salt may vary from 10% to 40% of the weight of the total composition or tablet.

The organic carboxylic acid required in the controlled release formulation of this present invention is in an amount which is enough to create a micro-environment of low pH of less than 7 in the vicinity of the dosage form hydrated. The ratio of the main drug to the acid may vary from 1:1 to 6:1. The ratio of 4:1 is preferably used. The acid is preferably an aliphatic organic carboxylic acid having from 3 to 20 carbon atoms, such as succinic acid, tartaric acid, maleic acid, glutaric acid, malic acid, glutamic acid, citric acid, mandelic acid, etc.. The most preferred acid is citric acid.

In the gastric fluid of the gastrointestinal tract, sodium alginate in the controlled release formulation will be converted into alginic acid. Alginic acid is slightly soluble in water and can form a film on the surface of the controlled release dosage form so as to control the drug release in the gastrointestinal tract, that is, the film can control the diffusion of the drug. In consequence, the insoluble alginate salts, such as sodium calcium alginate, are no longer required for the controlled release of clarithromycin.

Other inactive ingredients used in the controlled release formulation of the present invention include pharmaceutically acceptable excipients, diluents, preservatives, lubricants or glidants, and approved coloring agents. The dosage form may also be coated with materials not specifically designed for control or modification of drug release.

### EXAMPLE 1

1. Granulation for the Controlled Release Tablets

All tablet formulations were prepared by the following general manufacturing method. Clarithromycin, sodium alginate, lactose and stearic acid were passed through a # 40 mesh screen to remove any large agglomerates. The screened materials were mixed in a mixer (Supermixer) for 20 minutes. The resultant mixture was granulated with povidone and citric acid aqueous solution in suitable amounts until a suitable granulated mass was obtained. The wet mass was passed through a # 16 mesh screen and dried in a hot air oven at 60° C. until the granules had a moisture content of between 5% and 7%, determined by the Karl Fisher method. The dried granule was passed through a # 20 mesh screen and blended with tablet lubricants for 2 minutes by a V-Blender.

2. Compression

Tablets were compressed using a rotary tablet machine using oval type punches. Individual formulations I, II, and III were compressed to tablets having suitable thickness and friability. The compositions of the tablet formulations I, II and III are shown in the following Table I.

TABLE I

| | Formulae | | |
|---|---|---|---|
| | I | II | III |
| Ingredients | mg/tablet | mg/tablet | mg/tablet |
| Clarithromycin | 500 | 500 | 500 |
| Citric Acid Monohydrate | 110 | 120 | 125 |
| Sodium Alginate | 250 | 250 | 245 |
| Lactose Monohydrate | 88 | 78 | 78 |
| Povidone K 30 | 12 | 12 | 12 |
| Stearic acid | 5 | 5 | 5 |
| Talc | 5 | 5 | 5 |
| Magnesium stearate | 10 | 10 | 10 |

3. Film Coating

Tablets were coated by an aqueous suspension which contains hydroxypropyl methyl cellulose, polyethylene glycols, sorbic acid, titanium dioxide and purified water to increase average 2.5% of the tablet weight.

### EXAMPLE 2

### Dissolution Study

1. Dissolution Study According to USP 27, Page 463 (2004)

0.1 M sodium acetate buffer: obtained by transferring 13.61 g of sodium acetate trihydrate to a 1-liter volumetric flask, adding water to dissolve the trihydrate, diluting with water to the volume, mixing, and adjusting the pH to 5.0 with 0.1 M acetic acid.

Dissolution Medium: 900 mL of the abovesaid 0.1 M sodium acetate buffer.

Apparatus 2: 50 rpm.

Sampling timing: at hour 1, 2, 4, 6, 8, 10, 12, 18 and 24.

Mobile phase: obtained by preparing a mixture of methanol and 0.067 M monobasic potassium phosphate (650:350), adjusting with phosphoric acid to a FH of 4.0, filtering through a filter having a porosity of 0.5 μm or finer, and degassing, and making adjustments if necessary.

Standard preparation: obtained by dissolving about 27 mg of an accurately weighed quantity of clarithromycin in 40 mL of dissolution medium, shaking and sonicating if necessary to assure dissolution in a 50-mL volumetric flask, diluting with dissolution medium to volume, and mixing; filtering through a filter having a porosity of 0.5 μm or finer, and using the filtrate as the standard preparation. This solution contains about 54 μg of clarithromycin per mL.

Chromatographic system: the liquid chromatograph is equipped with a 210-nm detector, a 4.6-mm×15-cm Inertsil ODS-2: 5 μm, and is maintained at a constant temperature of about 50° C. The flow rate is about 1 mL per minute. Chromatograph the standard preparation and the test solution, and record the responses: the column efficiency, determined from clarithromycin peak, is not less than 750 theoretical plates, the tailing factor is not less than 0.9 and not more than 2, and relative standard deviation for replicate injections is not more than 2.0%.

Procedure: Separately inject equal volumes (about 20 to 50 μL) of the standard preparation and the test solution (i.e., a filtrate of the sample preparation diluted quantitatively with the mobile phase and initially containing about 54 μg of clarithromycin per mL.) into the chromatograph, record the chromatograms, and measure the responses for the major peaks. Calculate the quantity, in μg, of clarithromycin dissolved, by the formula:
900(CD)(rᵤ/rₛ),

wherein

C is the concentration of the standard preparation;

D is the appropriate dilution factor used to prepare the test solution; and

rᵤ and rₛ are the responses of the standard preparation and the test solution, respectively.

2. Dissolution Results and Discussion

The dissolution profiles of 6 tablets of the Klaricid® XL tablets (clarithromycin, 500 mg) from Abbott Laboratories Ltd., Queenborough, Kent, ME11 5EL, UK, and of 6 tablets of the present invention (clarithromycin sustained release tablets) were performed in 0.1 M sodium acetate buffer and assayed as described in the above dissolution study section. The dissolution results for the two different sources are as shown in Table II.

TABLE II
Dissolution study of the Clarithromycin XL tablets

| | Hr | | | | | | | | |
|---|---|---|---|---|---|---|---|---|---|
| | 1 | 2 | 4 | 6 | 8 | 10 | 12 | 18 | 24 |
| Product | Dissolution profile (average), in % | | | | | | | | |
| Klaricid ® XL | 3.52 | 9.24 | 21.87 | 36.09 | 46.33 | 55.75 | 62.25 | 76.47 | 86.41 |
| Invention | 4.27 | 9.77 | 23.29 | 37.53 | 49.53 | 58.80 | 66.41 | 81.93 | 90.53 |

Invention: Clarithromycin sustained release tablets of the present invention.

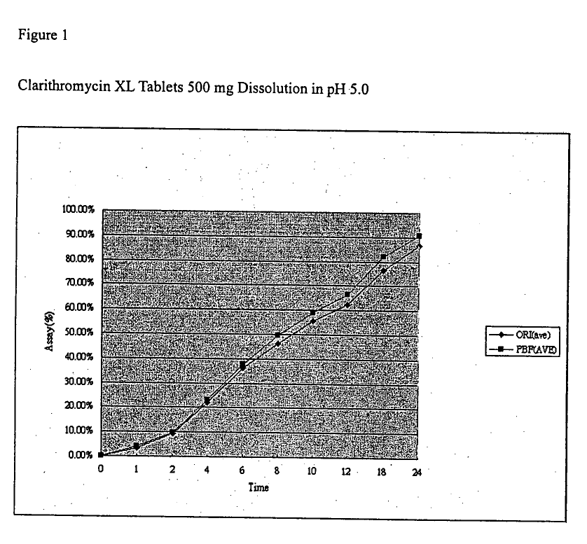

The dissolution profiles of the Klaricid® XL tablets (ORI) and of the clarithromycin sustained release tablets of the present invention (PBF) are shown in FIG. 1.

TABLE III
The dissolution results of each tablet from the Klaricid ® XL tablets (ORI)
and the average (ave) and relative standard deviation (RSD) in six runs.

| | | | | | | | ORI | ORI | ORI |
|---|---|---|---|---|---|---|---|---|---|
| Hr | ORI-1 | ORI-2 | ORI-3 | ORI-4 | ORI-5 | ORI-6 | (ave) | (SD) | (RSD) |
| 0 | 0 | 0 | 0 | 0 | 0 | 0 | 0 | 0 | 0 |
| 1 | 3.41% | 3.09% | 3.56% | 2.90% | 3.79% | 4.35% | 3.52% | 0.00518 | 14.73% |
| 2 | 9.91% | 8.51% | 9.50% | 8.06% | 9.20% | 10.22% | 9.24% | 0.008235 | 8.92% |
| 4 | 23.35% | 21.62% | 20.61% | 20.31% | 21.87% | 23.43% | 21.87% | 0.013195 | 6.03% |
| 6 | 37.01% | 35.82% | 36.44% | 34.11% | 35.80% | 37.34% | 36.09% | 0.011507 | 3.19% |
| 8 | 47.44% | 45.98% | 45.35% | 45.30% | 46.39% | 47.54% | 46.33% | 0.009818 | 2.12% |
| 10 | 56.36% | 54.77% | 57.72% | 54.81% | 55.08% | 55.77% | 55.75% | 0.011439 | 2.05% |
| 12 | 62.62% | 60.29% | 64.72% | 61.83% | 61.70% | 62.37% | 62.25% | 0.014533 | 2.33% |
| 18 | 74.38% | 75.13% | 78.24% | 76.52% | 77.40% | 77.13% | 76.47% | 0.014559 | 1.90% |
| 24 | 86.03% | 85.09% | 86.63% | 85.70% | 87.20% | 87.81% | 86.41% | 0.010007 | 1.16% |

TABLE IV
The dissolution results of each tablet from the Clarithromycin Sustained
Release tablets of the present invention (PBF) and the average (AVE) and relative
standard deviation (RSD) in six runs.

| | | | | | | | PBF | PBF | PBF |
|---|---|---|---|---|---|---|---|---|---|
| Hr | PBF-1 | PBF-2 | PBF-3 | PBF-4 | PBF-5 | PBF-6 | (AVE) | (SD) | (RSD) |
| 0 | 0 | 0 | 0 | 0 | 0 | 0 | 0 | 0 | 0 |
| 1 | 3.89% | 3.85% | 5.14% | 4.52% | 4.04% | 4.17% | 4.27% | 0.004478 | 10.49% |
| 2 | 9.48% | 9.21% | 10.71% | 9.89% | 9.64% | 9.68% | 9.77% | 0.304692 | 4.80% |
| 4 | 22.92% | 23.08% | 24.49% | 23.15% | 23.04% | 23.07% | 23.29% | 0.905403 | 2.32% |
| 6 | 37.06% | 37.73% | 38.73% | 38.07% | 37.04% | 36.53% | 37.53% | 0.00734 | 1.96% |
| 8 | 48.82% | 50.04% | 50.79% | 50.31% | 48.91% | 48.31% | 49.53% | 0.008975 | 1.81% |
| 10 | 57.70% | 59.66% | 60.41% | 59.39% | 58.07% | 57.54% | 58.80% | 0.010809 | 1.84% |
| 12 | 65.49% | 66.82% | 67.92% | 67.25% | 65.76% | 65.20% | 66.41% | 0.009905 | 1.49% |
| 18 | 81.50% | 82.17% | 83.82% | 82.06% | 81.29% | 80.71% | 81.93% | 0.009772 | 1.19% |
| 24 | 90.23% | 90.25% | 92.38% | 90.29% | 90.13% | 89.91% | 90.53% | 0.00836 | 0.92% |

The dissolution profiles of the Klaricid® XL tablets and of the Clarithromycin Sustained Release tablets of the present invention are similar, as shown in FIG. 1. The difference between the profiles of the Klaricid® XL tablets and Clarithromycin Sustained release Tablets of the present invention at each time period is less than 5%, except the difference of 5.46% (i.e., 81.93%-76.47%=5.46%) at Hr 18. These two types of tablets may be bioequivalent to each other in the clinical study.

In addition, the dissolution results of the Klaricid® XL tablets and Clarithromycin Sustained Release tablets of the present invention at each time period are shown in Table III and Table IV. The relative standard deviation (RSD) of between 1.16% and 14.73% in six runs for the Klaricid® XL tablets at each time period is much greater than the RSD of between 0.92% and 10.49% in six runs for Clarithromycin Sustained Release tablets of the present invention. The average RSDs from the Klaricid® XL tablets and the Clarithromycin Sustained Release tablets of the present invention are 4.71% and 2.98%, respectively. This indicates that the differences between the dissolution behaviors of the Clarithromycin Sustained Release tablets of the present invention are very small as compared with the differences between the dissolution behaviors of the Klaricid® XL tablets. This advantage achieved in the present invention is due to the use of sodium alginate alone in the Clarithromycin Sustained Release tablets instead of the use of both sodium alginate and sodium-calcium alginate in the Klaricid® XL tablets. Using both of two different-alginates may cause difficulty in the mixing step of the manufacturing process.

### The Advantages of the Present Invention

A. In the present invention, the method for producing the special pharmaceutical preparations of the present invention is easier to operate and can reduce the cost, as compared with the method disclosed in U.S. Pat. No. 5,705,190 in which two different alginates are used in the sustained release tablets.

B. In the gastric fluid of the gastrointestinal tract, sodium alginate in the tablets of the present invention will be converted into alginic acid which is slightly soluble in water and can form a film on the surface of the tablets so as to control the drug release in the gastrointestinal tract.

C. It is seen from the data in Table II that there are no significant differences between the tablets of the present invention and the Klaricid® XL tablets in their dissolution profiles. However, it is seen from Tables III and IV that the average RSD (relative standard deviation) in Table IV is lower than the average RSD in Table III, and this indicates that the differences between the dissolution behaviors of the Clarithromycin Sustained Release tablets of the present invention are very small as compared with the differences between the dissolution behaviors of the Klaricid® XL tablets.

1. A sustained release, solid pharmaceutical preparation for oral use comprising:
a therapeutically effective amount of at least one main drug which is sparingly water-soluble;
a water soluble alginate salt; and
an organic carboxylic acid to facilitate dissolution of the main drug;
wherein the weight ratio of the main drug to the organic carboxylic acid is within the range of from 1:1 to 6:1.

2. A pharmaceutical preparation as claimed in claim 1, which is in tablet form.

3. A pharmaceutical preparation as claimed in claim 1, which is in the dosage form suitable to be administered once a day.

4. A pharmaceutical preparation as claimed in claim 1, wherein the main drug is a macrolide antibiotic agent.

5. A pharmaceutical preparation as claimed in claim 4, wherein the macrolide antibiotic agent is clarithromycin.

6. A pharmaceutical preparation as claimed in claim 1, wherein the water soluble alginate salt is sodium alginate.

7. A pharmaceutical preparation as claimed in claim 1, wherein the organic carboxylic acid is selected from the group consisting of succinic acid, tartaric acid, maleic acid, glutaric acid, malic acid, glutamic acid, citric acid and mandelic acid.

8. A pharmaceutical preparation as claimed in claim 7, wherein the organic carboxylic acid is citric acid.

9. A pharmaceutical preparation as claimed in claim 6, wherein the weight ratio of sodium alginate to the main drug is about 1:2.

10. A pharmaceutical preparation as claimed in claim 1, wherein the main drug is selected from the group consisting of sulfamethoxazole, metronidazole, cimetidine, indapamide, atenolol, diazepam, omeprazole and ranitidine.

11. A pharmaceutical preparation as claimed in claim 4, wherein the macrolide antibiotic agent is selected from the group consisting of erythromycin, dirithromycin, azithromycin and roxithroniycin.

12. A pharmaceutical preparation as claimed in claim 1, which comprises about 500 mg of clarithromycin, from about 200 mg to 400 mg of sodium alginate, and from about 105 mg to 130 mg of citric acid.
